(19) United States
(12) Patent Application Publication
(10) Pub. No.: US 2019/0153570 A1
(43) Pub. Date: May 23, 2019

(54) FULLY DEGRADABLE MAGNESIUM ALLOY AND PREPARATION METHOD THEREOF

(71) Applicants: Qian ZHOU, Changsha (CN); Yugang JIANG, Changsha (CN)

(72) Inventors: Qian ZHOU, Changsha (CN); Yugang JIANG, Changsha (CN)

(21) Appl. No.: 15/781,151

(22) PCT Filed: Mar. 3, 2016
(86) PCT No.: PCT/CN2016/075396

§ 371 Date: Jun. 4, 2018

(30) Foreign Application Priority Data
Jan. 19, 2016 (CN) 201610033640.1

(51) Int. Cl. C22C 23/06; C22C 23/04; C22F 1/06

(52) U.S. Cl. CPC C22C 23/06; A61L 31/022; C22F 1/06; C22C 23/04

(57) ABSTRACT

Provided is a novel cardio-/cerebrovascular stent material of fully degradable magnesium alloy. The fully degradable magnesium alloy comprises magnesium and alloying elements, wherein the weight ratio of magnesium is not less than 85%, and the alloying elements include any one or a combination of several of gadolinium, erbium, thulium, yttrium, neodymium, holmium and zinc. The fully degradable magnesium alloy of the present invention has mechanical properties meeting the requirements of a cardio-/cerebrovascular biological stent, excellent corrosion resistance in vitro as demonstrated in in-vitro immersion corrosion test and electrochemical corrosion test, excellent biocompatibility as indicated in in-vitro cytotoxicity test, and a controllable degradation rate with good biocompatibility.

### CROSS REFERENCE TO RELATED APPLICATIONS

This application is the national phase entry of International Application No. PCT/CN2016/075396, filed on Mar. 3, 2016, which is based upon and claims priority to Chinese Patent Application No. 201610033640.1, filed on Jan. 19, 2016, the entire contents of which are incorporated herein by reference.

### TECHNICAL FIELD

The present invention relates to the field of biomaterials, and more specifically to a fully degradable magnesium alloy.

### BACKGROUND

Cardiovascular and cerebrovascular diseases have raised much attention due to their high morbidity and high mortality and disability rate. Stent placement and reconstruction of blood supply is an important method for clinical treatment of ischemic cardiovascular and cerebrovascular diseases. The main role of the stent in the blood vessel is to provide mechanical support to prevent elastic retraction and negative remodeling of the blood vessel. The diseased artery generally completes revascularization and repair within 6-12 months. After this time, the stent has no benefit to the human body and instead the pressure and stimulation to the blood vessel wall can create a series of problems. At present, the vascular stent materials used clinically at home and abroad are mainly 316L stainless steel, cobalt-chromium alloy and nickel-titanium alloy. These permanent metal stents have the following problems in the long-term implantation: long-term endothelial dysfunction and delayed endothelialization, procoagulant, long-term physical stimulation, local chronic inflammatory response, prolonged dual antiplatelet therapy, mismatched mechanical properties between the blood vessels around the stent and the normal blood vessels, occlusion of branch vessels, inconsistent with the growth and development of adolescents, and interference with CT and MR imaging, and more severely, the loss of chance of reoperation.

Ideal vascular stents should be able to degrade to be absorbed after repair of blood vessels. Compared with metal stents, degradable stents have obvious advantages: first, after the stents have degraded and been absorbed, there are no foreign substances remaining, reducing the risk factors that trigger thrombosis; second, they shorten the time for dual antiplatelet therapy and reduce bleeding and other related complications. Physiologically speaking, the disappearance of rigid stents is conducive to the restoration of vascular tone and remodeling. In the long term, degradable stents will not affect the follow-up treatment of coronary heart disease, such as PCI, coronary bypass or drug dissolving plaques. In addition, degradable stents do not interfere with CT or MR imaging and can eliminate the anxiety of a small number of patients carrying implants for life.

Due to the good biocompatibility and mechanical characteristics (strength, elasticity, ductility, stability) of magnesium metal, it has currently become a research hotspot for degradable scaffold materials. Biotronik Company in Germany has released two generations of AMS (absorb metal stent) blood vessel stents for clinical trials jointly in eight clinical centers in seven countries, including Australia, Germany, and Belgium, where 71 AMS stents have been successfully implanted, and found good safety, no death, no myocardial infarction, no thrombosis. The stents can be detected by MRI/CT and are completely degraded after four months.

The currently studied degradable materials of vascular stents mainly include polymer materials, ferroalloys, and magnesium alloys. Polymer materials are not developed under the X-rays, their radial support strength is insufficient, and their deformability is poor, which limit their application. The ferroalloy has a slow corrosion rate in the physiological environment, and the corrosion products block the blood vessels, so the ferroalloy is also not suitable for use in the degradable blood vessel stent. Due to its good biocompatibility and mechanical properties, magnesium metal has currently become a research hotspot for degradable stent materials. Internationally, only the vascular stent developed by using magnesium alloy WE43 by German scholars has currently entered the clinical trials. A multi-center randomized study found that the biodegradable magnesium alloy stent can achieve the same restoration of blood flow as the common metal stent in the early stage alter being implanted in patients with coronary artery stenosis, and the biodegradable magnesium alloy stent can be completely degraded after 4 months. In China, there is no clinical application report of magnesium alloy stents yet, only the institute of Metal Research of Chinese Academy of Sciences developed a magnesium alloy coronary stent of degradable magnesium alloy AZ31 series, and 12 stents were implanted into the abdominal aorta of 12 New Zealand white rabbits. During the follow-up period, the white rabbits survived well, the blood vessels in the stent implantation site kept patency, and no thrombosis was found. The stents were completely degraded after 4 months. The safety and effectiveness of the degradable magnesium alloy stents were confirmed from the animal experimental level. However, magnesium alloys used as degradable vascular stents still have some problems, such as rapid degradation resulting in rapid loss of strength, intimal hyperplasia resulting in narrowing of the lumen, low strength resulting in early rebound, and inexactness of biocompatibility of the magnesium alloy itself, etc., all of which need to be settled urgently.

### SUMMARY

The object of the present invention is to provide a fully degradable magnesium alloy and a preparation method thereof. The present invention develops a fully degradable magnesium alloy with more controllable degradation rate and better biocompatibility for the requirements of cardiovascular stents.

The present invention adopts the following technical solutions:

A fully degradable magnesium alloy including magnesium and alloying elements, in which a weight ratio of magnesium is not less than 85%, and the alloying elements include any one or a combination of several of gadolinium, erbium, thulium, yttrium, neodymium, holmium and zinc.

The weight ratios of gadolinium, erbium, thulium, yttrium, neodymium, holmium and zinc are at most 10.0%, 15.0%, 15.0%, 7.0%, 4.0%, 12.0% and 5.0%, respectively.

The weight ratios of gadolinium, erbium, and thulium are at least 0.1%, 0.1% and 0.1%, respectively.

Active elements are also included, which include any one or a combination of two of potassium, strontium, zirconium, calcium, lithium, aluminum and manganese.

The weight ratio of the active elements is at most 2%.

A method for preparing a fully degradable magnesium alloy includes the following steps:

adding a raw material to a resistance furnace for smelting to form a smelted material under a protective gas; refining the smelted material to form a refined material; cooling after pouring the refined material to form an ingot; proceeding with solid solution treatment on the ingot prior to plastic deformation for fining alloy crystal grains; and then performing heat treatment on the alloy crystal grains to obtain a fully degradable magnesium alloy billet.

The smelting temperature of the raw material is 720-820° C.

The pouring temperature of the refined material is 700-760° C.

The solid solution condition of the solid solution treatment 500-550° C. for 4-24 hours of treatment.

Magnesium, alloying elements and active elements, in which a weight ratio of magnesium is not less than 85%; and the alloying elements include any one or a combination of Several of gadolinium, erbium, thulium yttrium, neodymium, holmium and zinc. The active elements include any one or a combination of two of titanium, potassium, strontium, zirconium, calcium, lithium, aluminum and manganese, and a weight ratio of a content of the active elements is 0-2%; and magnesium, aluminum and zinc are added in a form of metal, and the other elements are added in a manner of an intermediate alloy.

The advantages of the present invention are as follows: the fully degradable magnesium alloy of the present invention has been confirmed by in-vitro immersion corrosion test and electrochemical corrosion test that its in-vitro corrosion resistance is similar to that of high-purity magnesium, and the in-vitro cytotoxicity test of the fully degradable magnesium alloy shows good biocompatibility, with controllable degradation rate and good biocompatibility.

### DETAILED DESCRIPTION OF THE EMBODIMENTS

Table 1 shows Embodiments 1-71 of a fully degradable magnesium alloy of the present invention, and a fully degradable magnesium alloy of the present invention can be obtained from the preparations.

Table 1 shows the components of Embodiments 1-71 of the present invention

| | Component (wt_%) | | | | | | | |
|---|---|---|---|---|---|---|---|---|
| Embodiments | Gadolinium | Erbium | Thulium | Yttrium | Neodymium | Holmium | Zinc | Magnesium |
| 1 | 10 | \ | \ | \ | \ | \ | \ | Balance |
| 2 | \ | 15 | \ | \ | \ | \ | \ | Balance |
| 3 | \ | \ | 15 | \ | \ | \ | \ | Balance |
| 4 | \ | \ | \ | 7 | \ | \ | \ | Balance |
| 5 | \ | \ | \ | \ | 4 | \ | \ | Balance |
| 6 | \ | \ | \ | \ | \ | 12 | \ | Balance |
| 7 | \ | \ | \ | \ | \ | \ | 5 | Balance |
| 8 | 10 | | | | 4 | \ | \ | Balance |
| 9 | 10 | \ | \ | \ | \ | | 5 | Balance |
| 10 | \ | \ | \ | 7 | 4 | \ | \ | Balance |
| 11 | \ | \ | \ | 7 | \ | \ | 5 | Balance |
| 12 | 10 | 4 | \ | \ | \ | \ | \ | Balance |
| 13 | 10 | \ | 4 | \ | \ | \ | \ | Balance |
| 14 | 10 | \ | \ | 4 | \ | \ | \ | Balance |
| 15 | 10 | \ | \ | \ | \ | 4 | \ | Balance |
| 16 | 10 | \ | \ | \ | \ | | 4 | Balance |
| 17 | 8 | \ | \ | 7 | \ | \ | \ | Balance |
| 18 | \ | 8 | \ | 7 | \ | \ | \ | Balance |
| 19 | \ | \ | 8 | 7 | \ | \ | \ | Balance |
| 20 | \ | \ | \ | 7 | 4 | \ | \ | Balance |
| 21 | \ | \ | \ | 7 | \ | 8 | \ | Balance |
| 22 | \ | \ | \ | 7 | \ | \ | 5 | Balance |
| 23 | \ | 11 | \ | \ | 4 | \ | \ | Balance |
| 24 | \ | \ | 11 | \ | 4 | \ | \ | Balance |
| 25 | \ | \ | \ | \ | 4 | 11 | | Balance |
| 26 | \ | \ | \ | \ | 4 | \ | 5 | Balance |
| 27 | 3 | \ | \ | \ | \ | 12 | \ | Balance |
| 28 | \ | 3 | \ | \ | \ | 12 | \ | Balance |
| 29 | \ | \ | 3 | \ | \ | 12 | \ | Balance |
| 30 | \ | \ | \ | 3 | \ | 12 | \ | Balance |
| 31 | \ | \ | \ | \ | 3 | 12 | | Balance |
| 32 | \ | \ | \ | \ | \ | 12 | 3 | Balance |
| 33 | \ | 10 | \ | \ | \ | \ | 5 | Balance |
| 34 | \ | \ | 10 | \ | \ | \ | 5 | Balance |
| 35 | \ | \ | \ | \ | \ | 10 | 5 | Balance |
| 36 | 0.1 | \ | \ | \ | \ | \ | \ | Balance |
| 37 | \ | 0.1 | \ | \ | \ | \ | \ | Balance |
| 38 | \ | \ | 0.1 | \ | \ | \ | \ | Balance |
| 39 | \ | \ | \ | \ | \ | 0.1 | \ | Balance |
| 40 | 4 | 7 | 4 | \ | \ | \ | \ | Balance |
| 41 | 4 | 7 | \ | 4 | \ | \ | \ | Balance |
| 42 | 3 | 8 | \ | \ | 4 | \ | \ | Balance |
| 43 | 2 | 9 | \ | \ | \ | 4 | \ | Balance |
| 44 | 5 | 6 | \ | \ | \ | \ | 4 | Balance |
| 45 | \ | 7 | 6 | 2 | \ | \ | \ | Balance |
| 46 | \ | 6 | 7 | \ | 2 | \ | \ | Balance |
| 47 | \ | 5 | 8 | \ | \ | 2 | \ | Balance |
| 48 | \ | 4 | 9 | \ | \ | \ | 2 | Balance |
| 49 | \ | \ | 7 | 3 | \ | 5 | \ | Balance |
| 50 | \ | \ | 4 | 3 | \ | \ | 3 | Balance |
| 51 | \ | \ | \ | 4 | 2 | \ | 3 | Balance |
| 52 | \ | \ | \ | 3 | 3 | 6 | \ | Balance |
| 53 | 2 | 3 | 4 | 1 | \ | \ | \ | Balance |
| 54 | 2 | 3 | 4 | \ | 5 | \ | \ | Balance |
| 55 | 2 | 3 | 4 | \ | \ | 2 | \ | Balance |
| 56 | 2 | 3 | 4 | \ | \ | \ | 2 | Balance |
| 57 | \ | 4 | 3 | 2 | 2 | \ | | Balance |
| 58 | \ | 3 | 4 | 2 | \ | \ | 1 | Balance |
| 59 | \ | \ | 5 | 2 | 3 | \ | 2 | Balance |
| 60 | \ | \ | 4 | 1 | 2 | 1 | \ | Balance |
| 61 | \ | \ | | 2 | 3 | 4 | 1 | Balance |
| 62 | 0.1 | 0.1 | 0.1 | 1 | 1 | \ | \ | Balance |
| 63 | 0.1 | 0.1 | 0.2 | 2 | \ | 1 | \ | Balance |
| 64 | 0.2 | 0.2 | 0.3 | 2 | \ | \ | 2 | Balance |
| 65 | 0.2 | 0.2 | 0.2 | 3 | 2 | 0.5 | \ | Balance |
| 66 | 0.3 | 0.1 | 0.1 | 2 | 1 | \ | 0.2 | Balance |
| 67 | \ | 0.5 | 0.2 | 3 | 2 | 0.5 | 0.2 | Balance |
| 68 | 0.5 | \ | 0.2 | 3 | 2 | 0.5 | 0.2 | Balance |
| 69 | 0.5 | 0.2 | \ | 3 | 2 | 0.5 | 0.2 | Balance |
| 70 | 0.5 | 3 | 0.2 | 2 | \ | 0.5 | 0.2 | Balance |
| 71 | 0.8 | 0.8 | 0.7 | 0.6 | 1 | 0.5 | 0.3 | Balance |

Table 2 shows Embodiments 72-110 of a fully degradable magnesium alloy of the present invention, and a fully degradable magnesium alloy of the present invention can be obtained by the preparations.

Table 2 shows the components of Embodiments 72-110 of the present invention

| | Component (wt_%) | | | | | | | |
|---|---|---|---|---|---|---|---|---|
| Embodiments | Gadolinium | Erbium | Thulium | Yttrium | Neodymium | Holmium | Zinc | Magnesium |
| 72 | 10 | \ | \ | \ | \ | \ | \ | Balance |
| 73 | \ | 13 | \ | \ | \ | \ | \ | Balance |
| 74 | \ | \ | 13 | \ | \ | \ | \ | Balance |
| 75 | \ | \ | \ | 7 | \ | \ | \ | Balance |
| 76 | \ | \ | \ | \ | 4 | \ | \ | Balance |
| 77 | \ | \ | \ | \ | \ | 12 | \ | Balance |
| 78 | \ | \ | \ | \ | \ | \ | 5 | Balance |
| 79 | 10 | \ | \ | \ | \ | \ | \ | Balance |
| 80 | \ | 13 | \ | \ | \ | \ | \ | Balance |
| 81 | \ | \ | 13 | \ | \ | \ | \ | Balance |
| 82 | \ | \ | \ | 7 | \ | \ | \ | Balance |
| 83 | \ | \ | \ | \ | 4 | \ | \ | Balance |
| 84 | \ | \ | \ | \ | \ | 12 | \ | Balance |
| 85 | \ | \ | \ | \ | \ | \ | 5 | Balance |
| 86 | 0.1 | 0.1 | 0.1 | 1 | 1 | \ | \ | Balance |
| 87 | 0.1 | 0.1 | 0.2 | 2 | \ | 1 | \ | Balance |
| 88 | 0.2 | 0.2 | 0.3 | 2 | \ | \ | 2 | Balance |
| 89 | 0.2 | 0.2 | 0.2 | 3 | 2 | 0.5 | \ | Balance |
| 90 | 0.3 | 0.1 | 0.1 | 2 | 1 | \ | 0.2 | Balance |
| 91 | \ | 0.5 | 0.2 | 3 | 2 | 0.5 | 0.2 | Balance |
| 92 | 0.5 | \ | 0.2 | 3 | 2 | 0.5 | 0.2 | Balance |
| 93 | 0.5 | 0.2 | \ | 3 | 2 | 0.5 | 0.2 | Balance |
| 94 | 0.5 | 3 | 0.2 | 2 | \ | 0.5 | 0.2 | Balance |
| 95 | 0.8 | 0.8 | 0.7 | 0.6 | 1 | 0.5 | 0.3 | Balance |
| 96 | 5.25 | \ | \ | 3.17 | 1.03 | \ | \ | Balance |
| 97 | 7.65 | \ | \ | 4.17 | 1.08 | \ | \ | Balance |
| 98 | 2.07 | \ | \ | 6.34 | 0.91 | \ | \ | Balance |
| 99 | \ | 3.72 | \ | 4.31 | 0.12 | \ | \ | Balance |
| 100 | 3.02 | 5.21 | \ | \ | \ | \ | \ | Balance |
| 101 | 2.78 | \ | \ | 1.55 | \ | \ | \ | Balance |
| 102 | 6.05 | \ | \ | 3.6 | \ | \ | \ | Balance |
| 103 | 3.05 | \ | \ | \ | 0.97 | \ | \ | Balance |
| 104 | 6.05 | \ | \ | \ | 1.79 | \ | \ | Balance |
| 105 | 10.17 | \ | \ | \ | 3.72 | \ | \ | Balance |
| 106 | 3.07 | \ | \ | \ | \ | 4.12 | \ | Balance |
| 107 | 6.51 | \ | \ | \ | \ | 7.31 | \ | Balance |
| 108 | 3.71 | \ | \ | \ | \ | \ | 1.17 | Balance |
| 109 | 9.05 | \ | \ | \ | \ | \ | 4.31 | Balance |
| 110 | 5 | \ | | 3 | 1 | \ | \ | Balance |
| | Component (wt_%) | | | | | | | |
| Embodiments | Titanium | Potassium | Strontium | Zirconium | Calcium | Lithium | Aluminum | Manganese |
| 72 | 2 | \ | \ | \ | \ | \ | \ | \ |
| 73 | \ | 2 | \ | \ | \ | \ | \ | \ |
| 74 | \ | \ | 2 | \ | \ | \ | \ | \ |
| 75 | \ | \ | \ | 2 | \ | \ | \ | \ |
| 76 | \ | \ | \ | \ | 2 | \ | \ | \ |
| 77 | \ | \ | \ | \ | \ | 2 | \ | \ |
| 78 | \ | \ | \ | \ | \ | \ | 2 | \ |
| 79 | \ | \ | \ | \ | \ | \ | \ | 2 |
| 80 | \ | \ | \ | \ | \ | \ | \ | 2 |
| 81 | 1 | 1 | \ | \ | \ | \ | \ | \ |
| 82 | 1 | | 1 | \ | \ | \ | \ | \ |
| 83 | 1 | \ | \ | 1 | \ | \ | \ | \ |
| 84 | 1 | \ | \ | \ | 1 | \ | \ | \ |
| 85 | 1 | \ | \ | \ | \ | 1 | \ | \ |
| 86 | 1 | \ | \ | \ | \ | \ | 1 | |
| 87 | 1 | \ | \ | \ | \ | \ | \ | 1 |
| 88 | \ | 0.3 | 0.5 | \ | \ | \ | \ | \ |
| 89 | \ | 0.3 | \ | \ | 0.2 | \ | \ | \ |
| 90 | \ | 0.3 | \ | \ | \ | 0.1 | \ | \ |
| 91 | \ | 0.3 | \ | \ | \ | \ | 0.3 | \ |
| 92 | \ | 0.3 | \ | \ | \ | \ | \ | 0.4 |
| 93 | \ | \ | 0.2 | \ | 0.2 | \ | \ | \ |
| 94 | \ | \ | \ | 0.2 | \ | 0.2 | \ | \ |
| 95 | \ | \ | \ | \ | 0.1 | \ | 0.4 | \ |
| 96 | \ | \ | \ | 0.42 | \ | \ | \ | \ |
| 97 | \ | \ | \ | 0.46 | \ | \ | \ | \ |
| 98 | \ | \ | \ | 0.46 | \ | \ | \ | \ |
| 99 | \ | \ | \ | 0.41 | \ | \ | \ | \ |
| 100 | \ | \ | | 0.41 | \ | \ | \ | \ |
| 101 | \ | \ | | 0.51 | \ | \ | \ | \ |
| 102 | \ | \ | | 0.41 | \ | \ | \ | \ |
| 103 | \ | \ | \ | 0.37 | \ | \ | \ | \ |
| 104 | \ | \ | \ | 0.39 | \ | \ | \ | \ |
| 105 | \ | \ | \ | 0.42 | \ | \ | \ | \ |
| 106 | \ | \ | \ | \ | \ | \ | \ | \ |
| 107 | \ | \ | \ | 0.36 | \ | \ | \ | \ |
| 108 | \ | \ | \ | 0.42 | \ | \ | \ | \ |
| 109 | \ | \ | \ | 0.51 | \ | \ | \ | \ |
| 110 | \ | \ | \ | 0.6 | \ | \ | \ | \ |

Table 3 shows Embodiments 111-135 of a fully degradable magnesium alloy of the present invention, and a fully degradable magnesium alloy of the present invention can be obtained by the preparations.

Table 3 shows the components of Embodiments 111-135 of the present invention

| | Component (wt_%) | | | | | | | | | | | | |
|---|---|---|---|---|---|---|---|---|---|---|---|---|---|
| Embod- | Gado- | | | | Neo- | | | Mag- | Titan- | Potas- | Stron- | Zirco- | |
| iments | linium | Erbium | Thulium | Yttrium | dymium | Holmium | Zinc | nesium | ium | sium | tium | nium | Calcium |
| 111 | 0.1 | 0.1 | 0.1 | 1 | 1 | \ | \ | Balance | 1 | \ | \ | \ | \ |
| 112 | 0.1 | 0.1 | 0.2 | 2 | \ | 1 | \ | Balance | 1 | \ | \ | \ | \ |
| 113 | 0.2 | 0.2 | 0.3 | 2 | \ | \ | 2 | Balance | \ | 0.3 | 0.5 | \ | \ |
| 114 | 0.2 | 0.2 | 0.2 | 3 | 2 | 0.5 | \ | Balance | \ | 0.3 | \ | \ | 0.2 |
| 115 | 0.3 | 0.1 | 0.1 | 2 | 1 | \ | 0.2 | Balance | \ | 0.3 | \ | \ | \ |
| 116 | \ | 0.5 | 0.2 | 3 | 2 | 0.5 | 0.2 | Balance | \ | 0.3 | \ | \ | \ |
| 117 | 0.5 | \ | 0.2 | 3 | 2 | 0.5 | 0.2 | Balance | \ | 0.3 | \ | \ | \ |
| 118 | 0.5 | 0.2 | \ | 3 | 2 | 0.5 | 0.2 | Balance | \ | \ | 0.2 | \ | 0.2 |
| 119 | 0.5 | 3 | 0.2 | 2 | \ | 0.5 | 0.2 | Balance | \ | \ | \ | 0.2 | \ |
| 120 | 0.8 | 0.8 | 0.7 | 0.6 | 1 | 0.5 | 0.3 | Balance | \ | \ | \ | \ | 0.1 |
| 121 | 5.25 | \ | \ | 3.17 | 1.03 | \ | \ | Balance | \ | \ | \ | 0.42 | \ |
| 122 | 7.65 | \ | \ | 4.17 | 1.08 | \ | \ | Balance | \ | \ | \ | 0.46 | \ |
| 123 | 2.07 | \ | \ | 6.34 | 0.91 | \ | \ | Balance | \ | \ | \ | 0.46 | \ |
| 124 | \ | 3.72 | \ | 4.31 | 0.12 | \ | \ | Balance | \ | \ | \ | 0.41 | \ |
| 125 | 3.02 | 5.21 | \ | \ | \ | \ | \ | Balance | \ | \ | | 0.41 | \ |
| 126 | 2.78 | \ | \ | 1.55 | \ | \ | \ | Balance | \ | \ | | 0.51 | \ |
| 127 | 6.05 | \ | \ | 3.6 | \ | \ | \ | Balance | \ | \ | | 0.41 | \ |
| 128 | 3.05 | \ | \ | \ | 0.97 | \ | \ | Balance | \ | \ | \ | 0.37 | \ |
| 129 | 6.05 | \ | \ | \ | 1.79 | \ | \ | Balance | \ | \ | \ | 0.39 | \ |
| 130 | 10.17 | \ | \ | \ | 3.72 | \ | \ | Balance | \ | \ | \ | 0.42 | \ |
| 131 | 3.07 | \ | \ | \ | \ | 4.12 | \ | Balance | \ | \ | \ | \ | \ |
| 132 | 6.51 | \ | \ | \ | \ | 7.31 | \ | Balance | \ | \ | \ | 0.36 | \ |
| 133 | 3.71 | \ | \ | \ | \ | \ | 1.17 | Balance | \ | \ | \ | 0.42 | \ |
| 134 | 9.05 | \ | \ | \ | \ | \ | 4.31 | Balance | \ | \ | \ | 0.51 | \ |
| 135 | 5 | 0.2 | 0.3 | 3 | 1 | 0.2 | 0.1 | Balance | 0.5 | 0.2 | 0.3 | 0.6 | 0.1 |

| | Component (wt_%) | | | | | | | | | | | |
|---|---|---|---|---|---|---|---|---|---|---|---|---|
| Embod- | Lith- | Alumi- | Manga- | Lantha- | | Praseo- | Prome- | Samar- | | Dyspro- | Ytter- | Lute- |
| iments | ium | num | nese | num | Cerium | dymium | thium | ium | Terbium | sium | bium | tium |
| 111 | \ | 1 | | 0.1 | 0.1 | 0.1 | 1 | 1 | \ | \ | 0.3 | |
| 112 | \ | \ | 1 | 0.1 | 0.1 | 0.2 | 2 | \ | 1 | \ | 0.5 | 1 |
| 113 | \ | \ | \ | 0.2 | 0.2 | 0.3 | 2 | \ | \ | 2 | 0.3 | 1 |
| 114 | \ | \ | \ | 0.2 | 0.2 | 0.2 | 3 | 2 | 0.5 | \ | 0.5 | \ |
| 115 | 0.1 | \ | \ | 0.3 | 0.1 | 0.1 | 2 | 1 | \ | 0.2 | 0.2 | \ |
| 116 | \ | 0.3 | \ | \ | 0.5 | 0.2 | 3 | 2 | 0.5 | 0.2 | 0.1 | \ |
| 117 | \ | \ | 0.4 | 0.5 | \ | 0.2 | 3 | 2 | 0.5 | 0.2 | 0.2 | \ |
| 118 | \ | \ | \ | 0.5 | 0.2 | \ | 3 | 2 | 0.5 | 0.2 | 0.3 | \ |
| 119 | 0.2 | \ | \ | 0.5 | 3 | 0.2 | 2 | \ | 0.5 | 0.2 | 0.3 | \ |
| 120 | \ | 0.4 | \ | 0.8 | 0.8 | 0.7 | 0.6 | 1 | 0.5 | 0.3 | 0.1 | \ |
| 121 | \ | \ | \ | 0.5 | \ | \ | 0.31 | 0.3 | \ | \ | 0.2 | \ |
| 122 | \ | \ | \ | 0.6 | \ | \ | 0.41 | 0.2 | \ | \ | 0.3 | \ |
| 123 | \ | \ | \ | 0.2 | \ | \ | 0.6 | 0.91 | \ | \ | 0.2 | \ |
| 124 | \ | \ | \ | \ | 0.3 | \ | 0.4 | 0.12 | \ | \ | 0.1 | \ |
| 125 | \ | \ | \ | 0.3 | 0.2 | \ | \ | \ | \ | \ | 0.3 | \ |
| 126 | \ | \ | \ | 0.2 | \ | \ | 0.2 | \ | \ | \ | 0.2 | \ |
| 127 | \ | \ | \ | 0.5 | \ | \ | 0.3 | \ | \ | \ | 0.2 | \ |
| 128 | \ | \ | \ | 0.3 | \ | \ | \ | 0.97 | \ | \ | 0.1 | \ |
| 129 | \ | \ | \ | 0.2 | \ | \ | \ | 0.2 | \ | \ | 0.2 | \ |
| 130 | \ | \ | \ | 0.5 | \ | \ | \ | 0.3 | \ | \ | 0.1 | \ |
| 131 | \ | \ | \ | 0.3 | \ | \ | \ | \ | 0.1 | \ | 0.2 | \ |
| 132 | \ | \ | \ | 0.2 | \ | \ | \ | \ | 0.2 | \ | 0.2 | \ |
| 133 | \ | \ | \ | 0.3 | \ | \ | \ | \ | \ | 0.1 | 0.3 | \ |
| 134 | \ | \ | \ | 0.9 | \ | \ | \ | \ | \ | 0.3 | 0.4 | \ |
| 135 | 0.1 | 0.3 | 0.2 | 0.5 | 0.1 | 0.1 | 3 | 1 | 0.2 | 0.1 | 0.2 | 0.2 |

The present invention further discloses a method for preparing a fully degradable magnesium alloy, comprising the following steps:

The raw materials are added to the resistance furnace for smelting. The process is carried out under protective gas. After refining, proceed with pouring and cooling to form ingots. Proceed with solid solution treatment prior to plastic deformation for fining the alloy crystal. grains, and then heat treatment is performed to obtain a fully degradable magnesium alloy billet. The smelting temperature of the alloy is 720-820° C. The pouring temperature of the alloy is 700-760° C. The solid solution condition is 500-550° C. for 4-24 hours of treatment. Magnesium, alloying elements and active elements, in which the weight ratio of magnesium is not less than 85%, and the alloying elements include any one or a combination of several of gadolinium, erbium, thulium, yttrium, neodymium, holmium and zinc. Active elements include any one or a combination of two of titanium, potassium, strontium, zirconium, calcium, lithium, aluminum and manganese, and the weight ratios of the content of the active elements are 0-2%; and magnesium, aluminum and zinc are added in the form of metal, and the other elements are added in the manner of an intermediate alloy

TABLE 4
Preparation process of a fully degradable magnesium alloy

| | Smelting | Pouring | Solid | |
|---|---|---|---|---|
| | temperature | temperature | solution | |
| Embodiment | (° C.) | (° C.) | treatment | Plastic deformation |
| Embodiment | 780 | 740 | 520° C./8 h | Extrusion at 400° C., |
| 96 | | | | extrusion ratio: 16, extrusion |
| | | | | speed: 10 mm/min |
| Embodiment | 760 | 745 | 520° C./8 h | Extrusion at 420° C., |
| 97 | | | | extrusion ratio: 16 extrusion |
| | | | | speed: 10 mm/min |
| Embodiment | 770 | 750 | 520° C./8 h | Extrusion at 380° C., |
| 98 | | | | extrusion ratio: 16, extrusion |
| | | | | speed: 10 mm/min |
| Embodiment | 765 | 760 | 520° C./8 h | Extrusion at 450° C., |
| 99 | | | | extrusion ratio: 16, extrusion |
| | | | | speed: 10 mm/min |
| Embodiment | 760 | 740 | 520° C./8 h | Extrusion at 400° C., |
| 100 | | | | extrusion ratio: 16 extrusion |
| | | | | speed: 10 mm/min |
| Embodiment | 770 | 740 | 520° C./8 h | Extrusion at 450° C., |
| 101 | | | | extrusion ratio: 16, extrusion |
| | | | | speed: 10 mm/min |
| Embodiment | 780 | 760 | 520° C./8 h | Extrusion at 450° C., |
| 102 | | | | extrusion ratio: rato: 16, extrusion |
| | | | | speed: 10 mm/min |
| Embodiment | 780 | 740 | 520° C./8 h | Extrusion at 450° C. |
| 103 | | | | extrusion ratio: 16, extuision |
| | | | | speed: 10 mm/min |
| Embodiment | 765 | 740 | 520° C./8 h | Extrusion at 450° C., |
| 104 | | | | extrusion ratio: 16, extrusion |
| | | | | speed; 10 mm/min |
| Embodiment | 780 | 740 | 520° C./8 h | Extrusion at 450° C., |
| 105 | | | | extrusion ratio: 16, extrusion |
| | | | | speed: 10 mm/min |
| Embodiment | 780 | 740 | 520° C./8 h | Extrusion at 450° C., |
| 106 | | | | extrusion ratio: 16, extrusion |
| | | | | speed: 10 mm/min |
| Embodiment | 780 | 740 | 520° C/8 h | Extrusion at 450° C., |
| 107 | | | | extrusion ratio: 16, extrusion |
| | | | | speed: 10 mm/min |
| Embodiment | 780 | 740 | 520° C./8 h | Extrusion at 450° C., |
| 108 | | | | extrusion ratio: 16, extrusion |
| | | | | speed: 10 mm/min |
| Embodiment | 780 | 740 | 520° C./8 h | Extrusion at 450° C., |
| 109 | | | | extrusion ratio: 16, extrusion |
| | | | | speed: 10 mm/min |

Immersion Corrosion Test and Results

TABLE 5
Mechanical properties of alloys with different components

| | Tensile | Yield | |
|---|---|---|---|
| Alloy Number | strength | strength | Elongation |
| Embodiment 96 | 270 | 203 | 15.2 |
| Embodiment 97 | 312 | 256 | 10.4 |
| Embodiment 98 | 252 | 207 | 10.2 |
| Embodiment 99 | 237 | 198 | 17.9 |
| Embodiment 100 | 249 | 202 | 19.1 |
| Embodiment 101 | 223 | 182 | 25.2 |
| Embodiment 102 | 265 | 213 | 17.0 |
| Embodiment 103 | 213 | 172 | 27.1 |
| Embodiment 104 | 245 | 201 | 22.5 |
| Embodiment 105 | 315 | 252 | 11.3 |
| Embodiment 106 | 261 | 204 | 18.2 |
| Embodiment 107 | 312 | 263 | 11.7 |
| Embodiment 108 | 211 | 165 | 24.7 |
| Embodiment 109 | 321 | 268 | 12.1 |

From the above table, it can be seen that the fully degradable magnesium alloys have excellent mechanical properties.

The fully degradable magnesium alloy of Embodiment 110 was subjected to an immersion corrosion test and an in-vitro cytotoxicity test.

Immersion Corrosion Test and Results

Immersion corrosion is performed according to the ASTM G31-72 standard. A fully degradable magnesium alloy metal sheet having a diameter of 8 mm and a thickness of 5 mm was sanded by 1200 Grit sandpaper until smooth, followed by ultrasonic cleaning in acetone, anhydrous ethanol, and distilled water, respectively. Record the weight and surface area of the metal, sterilize the fully degradable magnesium alloy cylindrical piece under UV light, and irradiate each side for 30 minutes. Put the metal piece into a test tube containing DMEM+10% FBS+1% penicillin/streptomycin, and the ratio of the solution volume to the metal surface area is 20 mL/cm². Place the test tube in an incubator of 37° C. 5% CO₂, take the test tube out after one week, two weeks and three weeks respectively wash the metal sheet with double distilled water, and dry at room temperature. The 200 g/L chromic acid is used to clean and remove the corrosion products deposited on the surface of the sample and the surface morphology of the sample is observed by scanning electron microscopy. According to ASTM G31-72 corrosion rate calculation formula: corrosion rate=(K×W)/(A×T×D) with the unit in mm/a, where K=8.76×10⁴, W is mass difference (g) between before and after immersion, A is the surface area of the sample in contact with the solution (cm²), T is the immersion time (h), and D is the sample density (g/cm³).

TABLE 6
In-vitro degradation rate of fully degradable magnesium alloy,
magnesium alloy AZ31 and pure magnesium (mm/a)

| | 1 Week | 2 Weeks | 3 Weeks |
|---|---|---|---|
| Fully degradable Mg alloy [symbol] | 0.330 ± 0.170 | 0.264 ± 0.082 | 0.247 ± 0.081 |
| Mg allow AZ231 [symbol] | 1.138 ± 0.741 | 0.695 ± 0.382 | 0.614 ± 0.221 |
| Pure Mg [symbol] | 0.259 ± 0.110 | 0.220 ± 0.076 | 0.205 ± 0.051 |

It can be seen from Table 6 that the degradation rates of the fully degradable magnesium alloy, magnesium alloy AZ31, and pure magnesium after having been immersed in the simulated body fluid for 1, 2, and 3 weeks are shown in Table 6. The results show that the degradation rates of the fully degradable magnesium alloy at three time points are much slower than that of magnesium alloy AZ31, but similar to that of pure magnesium. The in-vitro immersion corrosion test and electrochemical corrosion test of the fully degradable magnesium alloy confirmed that its in-vitro corrosion resistance is similar to that of high-purity magnesium and superior to that of magnesium alloy AZ31.

In-Vitro Cytotoxicity Test and Results

Set extract groups of different concentrations and negative and positive control groups. Add 10% FBS and 1% penicillin/streptomycin double antibiotics to RPMI 1 1640/DMEM medium (RPMI 1640 used to culture HUVEC-12, DMEM used to culture HASMC). Expose the fully degradable magnesium alloy cylindrical piece to ultraviolet light for irradiation and sterilization, irradiate each side for 30 minutes respectively. Then, place the magnesium alloy fully degradable magnesium alloy cylindrical piece in a test tube containing RPMI 1640 complete medium/DMEM complete medium. The ratio of the surface area of the sample to the volume of the culture medium is 1.25 cm²/ml. Place it in the incubator of 37° C., 95% relative humidity, 5% CO₂ for 72 hours, and then take out the magnesium alloy fully degradable magnesium alloy cylindrical piece to obtain the stock solution of the material extract (100% M), and dilute the extract with complete medium to 50% M, 25% M and 10% M. The HUVEC-12 cells and HASMC cells in the logarithmic growth phase are inoculated at a concentration of 3×10⁴ cells/ml in a 96-well flat-bottomed culture plate. Each group is performed in 5 wells in parallel, with 100 μL of cell suspension per well, a total of 6 groups and each kind of cells being inoculated on 2 plates. Place the 96-well flat-bottomed plates which had been inoculated with cells in the incubator of 37° C., 95% relative humidity, 5% CO₂ for 24 hours to allow the cells to grow adhering to the wall. Take out the 96-well culture plate, suct and discard the culture medium, and add extracts of different concentrations to the 96-well plate, 1640/DMEM complete medium to the negative control group and medium with 0.64% phenol to the positive control group. After addition of the liquids, place the 96-well culture plate in an incubator of 37° C., 5% CO₂ to incubate. Pick one 96-well culture plate each on the first and third days and add 10 uL MTT (5 mg/L) to each well. Continue the culture in the CO₂ incubator for 4 hours. The original culture medium containing MTT is then sucted and discarded, and add 150 μL of DMSO, shaking at room temperature and away from light on the shaker in a small amplitude for 10 minutes, so that the crystal is fully dissolved. The OD value of each well is measured with a microplate reader at a wavelength of 490 nm.

The formula for relative growth rate of the cell is:

RGR=(ODt/ODn)×100%

where ODt represents the average absorbance value of the experimental group, and ODn represents the average absorbance value of the negative control group.

Results.

TABLE 7
OD value and RGR of HASMC in different fully degradable magnesium alloy extracts

| Incubation | | OD | RGR | Cytotoxicity |
|---|---|---|---|---|
| Days | Groups | (χ̅ ± SD) | (%) | classification |
| 1 d | Negative control group | 0.5142 ± 0.04032 | 100 | 0 |
| | 100% extract group | 0.4383 ± 0.03480 | 85.24 | 1 |
| | 50% extract group | 0.4731 ± 0.02721 | 92.01 | 1 |
| | 25% extract group | 0.4701 ± 0.02947 | 91.42 | 1 |
| | 10% extract group | 0.4738 ± 0.03882 | 92.84 | 1 |
| | Positive control group | 0.1043 ± 0.00610 | 20.28 | 4 |
| 3 d | Negative control group | 0.4986 ± 0.01964 | 100 | 0 |
| | 100% extract group | 0.4154 ± 0.02053 | 83.31 | 1 |
| | 50% extract group | 0.4392 ± 0.03277 | 88.08 | 1 |
| | 25% extract group | 0.4476 ± 0.01895 | 89.77 | 1 |
| | 10% extract group | 0.4482 ± 0.02333 | 89.89 | 1 |
| | Positive control group | 0.0833 ± 0.00682 | 16.71 | 4 |

TABLE 8
OD value and RGR of HUVEC-12 in different fully degradable magnesium alloy extracts

| Incubation | | OD | RGR | Cytotoxicity |
|---|---|---|---|---|
| Days | Groups | (χ̅ ± SD) | (%) | classification |
| 1 d | Negative control group | 0.9552 ± 0.07621 | 100 | 0 |
| | 100% extract group | 0.7508 ± 0.04051 | 78.60 | 1 |
| | 50% extract group | 0.8235 ± 0.07417 | 86.21 | 1 |
| | 25% extract group | 0.8321 ± 0.06267 | 87.11 | 1 |
| | 10% extract group | 0.8579 ± 0.06334 | 89.81 | 1 |
| | Positive control group | 0.0522 ± 0.00173 | 5.46 | 4 |
| 3 d | Negative control group | 1.2084 ± 0.16370 | 100 | 0 |
| | 100% extract group | 0.9270 ± 0.0663 | 76.34 | 1 |
| | 50% extract group | 0.9422 ± 0.04390 | 79.32 | 1 |
| | 25% extract group | 1.0980 ± 0.05049 | 90.86 | 1 |
| | 10% extract group | 1.3140 ± 0.12878 | 108.73 | 0 |
| | Positive control group | 0.0860 ± 0.01190 | 7.12 | 4 |

The results of the experiments show that after the extracts of the fully degradable magnesium alloys have contacted with HASMC and HUVEC-12 cells respectively and incubated for 1 day and 3 days, the RGRs of the extract groups of different concentrations are greater than 75%, which are not significantly different from the negative control groups. The fully degradable magnesium alloy extracts show no toxic effect on the above two kinds of cells, no increase in toxicity with the prolonged incubation time, and no effect on the growth and proliferation of both cells. The cytotoxicity of the fully degradable magnesium alloy meets the requirements for biomedical materials used in vivo. The in-vitro cytotoxicity test of the fully degradable magnesium alloy shows good biocompatibility. It can be used as a preparation material for absorbable blood vessels and for medical equipments such as absorbable skull locks.

Immersion Corrosion Test and Results

TABLE 9
Mechanical properties of alloys with different components

| Alloy Number | Tensile strength | Yield strength | Elongation |
|---|---|---|---|
| Embodiment 135 | 335 | 278 | 14.2 |

From the above table, it can be seen that the fully degradable magnesium alloy has excellent mechanical properties.

The fully degradable magnesium alloy of Embodiment 135 is subjected to immersion corrosion test and in-vitro cytotoxicity test.

Immersion Corrosion Test and Results

Immersion corrosion is performed according to the ASTM G31-72 standard. A fully degradable magnesium alloy metal sheet having a diameter of 8 mm and a thickness of 5 mm was sanded by 1200 Grit sandpaper until smooth, followed by ultrasonic cleaning in acetone, anhydrous ethanol, and distilled water, respectively. Record the weight and surface area of the metal, sterilize the fully degradable magnesium alloy cylindrical piece under UV light, and irradiate each side for 30 minutes. Put the metal piece into a test tube containing DMEM+10% FBS+1% penicillin/streptomycin, and the ratio of the solution volume to the metal surface area is 20 mL/cm². Place the test tube in an incubator of 37° C., 5% CO₂, take the test tube out after one week, two weeks and three weeks respectively, wash the metal sheet with double distilled water, and dry at room temperature. The 200 g/L chromic acid is used to clean and remove the corrosion products deposited on the surface of the sample and the surface morphology of the sample is observed by scanning electron microscopy. According to ASTM G31-72 corrosion rate calculation formula: corrosion rate=(K×W)/(A×T×D) with the unit in mm/a, where K=8.76×10⁴, W is mass difference (g) between before and after immersion, A is the surface area of the sample in contact with the solution (cm²), T is the immersion time (h), and D is the sample density (g/cm³).

TABLE 10
In-vitro degradation rate of fully degradable magnesium alloy,
magnesium alloy AZ31 and pure magnesium (mm/a)

| | 1 Week | 2 Weeks | 3 Weeks |
|---|---|---|---|
| Fully degradable Mg alloy [symbol] | 0.261 ± 0.170 | 0.254 ± 0.082 | 0.237 ± 0.081 |
| Mg alloy AZ231 [symbol] | 1.138 ± 0.741 | 0.695 ± 0.382 | 0.614 ± 0.221 |
| Pure Mg [symbol] | 0.259 ± 0.110 | 0.220 ± 0.076 | 0.205 ± 0.051 |

It can be seen from Table 10 that the degradation rates of the fully degradable magnesium alloy, magnesium alloy AZ31, and pure magnesium after having been immersed in the simulated body fluid for 1, 2, and 3 weeks are shown in Table 10. The results show that the degradation rates of the fully degradable magnesium alloy at three time points are much slower than that of magnesium alloy AZ31, but similar to that of pure magnesium. The in-vitro immersion corrosion test and electrochemical corrosion test of the fully degradable magnesium alloy confirmed that its in-vitro corrosion resistance is similar to that of high-purity magnesium and superior to that of magnesium alloy AZ31.

In-Vitro Cytotoxicity Test and Results

Set extract groups of different concentrations and negative and positive control groups. Add 10% FBS and 1% penicillin/streptomycin double antibioticsto RPMI 1640/DMEM medium (RPMI 1640 used to culture HUVEC-12, DMEM used to culture HASMC). Expose the filly degradable magnesium alloy cylindrical piece to ultraviolet light for irradiation and sterilization, irradiate each side for 30 minutes respectively. Then, place the magnesium alloy fully degradable magnesium alloy cylindrical piece in a test tube containing RPMI 1640 complete medium/DMEM complete medium. The ratio of the surface area of the sample to the volume of the culture medium is 1.25 cm²/ml. Place it in the incubator of 37° C. 95% relative humidity, 5% CO₂ for 72 hours, and then take out the magnesium alloy fully degradable magnesium alloy cylindrical piece to obtain the stock solution of the raw material extract (100% M), and dilute the extract with complete medium to 50% M, 25% M and 10% M. The HUVEC-12 cells and HASMC cells in the logarithmic growth phase are inoculated at a concentration of 3×10⁴ cells/ml in a 96-well flat-bottomed culture plate. Each group is performed in 5 wells in parallel, with 100 μL of cell suspension per well, a total of 6 groups and each kind of cells being inoculated on 2 plates. Place the 96-well flat-bottomed plates which had been inoculated with cells in the incubator of 37° C., 95% relative humidity, 5% CO₂ for 24 hours to allow the cells to grow adhering to the wall. Take out the 96-well culture plate, suct and discard the culture medium, and add extracts of different concentrations to the 96-well plate, 1640/DMEM complete medium to the negative control group and medium with 0.64% phenol to the positive control group. After addition of the liquids, place the 96-well culture plate in an incubator of 37° C., 5% CO₂ to incubate. Pick one 96-well culture plate each on the first and third days and add 10 uL MTT (5 mg/L) to each well. Continue the culture in the CO₂ incubator for 4 hours. The original culture medium containing MTT is then sucted and discarded, and add 150 μL of DMSO, shaking at room temperature and away from light on the shaker in a small amplitude for 10 minutes, so that the crystal is fully dissolved. The OD value of each well is measured with a microplate reader at a wavelength of 490 nm.

The formula for relative growth rate of the cell is:

RGR=(ODt/ODn)×100%

where ODt represents the average absorbance value of the experimental group, and ODn represents the average absorbance value of the negative control group.

Results:

TABLE 11
OD value and RGR of HASMC in different fully degradable
magnesium alloy extracts

| Incu- | | | | |
|---|---|---|---|---|
| bation | | OD | RGR | Cytotoxicity |
| Days | Groups | (χ̅ ± SD) | (%) | classification |
| 1 d | Negative control group | 0.5142 ± 0.04032 | 100 | 0 |
| | 100% extract group | 0.4183 ± 0.03480 | 89.24 | 1 |
| | 50% extract group | 0.4231 ± 0.02721 | 93.01 | 1 |
| | 25% extract group | 0.4201 ± 0.02947 | 93.42 | 1 |
| | 10% extract group | 0.4238 ± 0.03882 | 93.84 | 1 |
| | Positive control group | 0.8043 ± 0.00510 | 15.28 | 4 |
| 3 d | Negative control group | 0.4986 ± 0.01964 | 100 | 0 |
| | 100% extract group | 0.3954 ± 0.02053 | 81.31 | 1 |
| | 50% extract group | 0.4092 ± 0.03277 | 85.08 | 1 |
| | 25% extract group | 0.4176 ± 0.01895 | 86.77 | 1 |
| | 10% extract group | 0.4082 ± 0.02333 | 87.89 | 1 |
| | Positive control group | 0.0783 ± 0.00682 | 14.71 | 4 |

TABLE 12
OD value and RGR of HUATEC-12 in different fully
degradable magnesium alloy extracts

| Incu- | | | | |
|---|---|---|---|---|
| bation | | OD | RGR | Cytotoxicity |
| Days | Groups | (χ̅ ± SD) | (%) | classification |
| 1 d | Negative control group | 0.9552 ± 0.07621 | 100 | 0 |
| | 100% extract group | 0.7308 ± 0.04051 | 78.60 | 1 |
| | 50% extract group | 0.8035 ± 0.07417 | 86.21 | 1 |
| | 25% extract group | 0.7921 ± 0.06267 | 87.11 | 1 |
| | 10% extract group | 0.8179 ± 0.06334 | 89.81 | 1 |
| | Positive control group | 0.0452 ± 0.00173 | 5.46 | 4 |
| 3 d | Negative control group | 1.2084 ± 0.16370 | 100 | 0 |
| | 100% extract group | 0.9070 ± 0.0663 | 76.34 | 1 |
| | 50% extract group | 0.9222 ± 0.04390 | 79.32 | 1 |
| | 25% extract group | 0.9822 ± 0.05049 | 90.86 | 1 |
| | 10% extract group | 1.1222 ± 0.12878 | 108.73 | 0 |
| | Positive control group | 0.0760 ± 0.01190 | 7.12 | 4 |

The results of the experiments show that after the extracts of the fully degradable magnesium alloys have contacted with HASMC and HUVEC-12 cells respectively and incubated for 1 day and 3 days, the RGRs of the extract groups of different concentrations are greater than 75%, which are not significantly different from the negative control groups. The fully degradable magnesium alloy extracts show no toxic effect on the above two kinds of cells, no increase in toxicity with the prolonged incubation time, and no effect on the growth and proliferation of both cells. The cytotoxicity of the fully degradable magnesium alloy meets the requirements for biomedical materials used in vivo. The in-vitro cytotoxicity test of the fully degradable magnesium alloy shows good biocompatibility. It can be used as a preparation material for absorbable blood vessels and for medical equipments such as absorbable skull locks, and the effect is better than that of the embodiment 110.

The foregoing descriptions are only preferred embodiments of the present invention and are not intended to limit the present invention. Any modification, equivalent replacement and improvement made within the spirit and principle of the present invention shall be included within the protection range of the present invention.

1. A fully degradable magnesium alloy, comprising magnesium and alloying elements, wherein a weight ratio of magnesium is not less than 85%, and the alloying elements include any one or a combination of several of gadolinium, erbium, thulium, yttrium, neodymium, holmium and zinc.

2. The fully degradable magnesium alloy according to claim 1, wherein weight ratios of gadolinium, erbium, thulium, yttrium, neodymium, holmium and zinc are at most 10.0%, 15.0%, 15.0%, 7.0%, 4.0%, 12.0% and 5.0%, respectively.

3. The fully degradable magnesium alloy according to claim 2, wherein the weight ratios of gadolinium, erbium, and thulium are at least 0.1%, 0.1% and 0.1%, respectively.

4. The fully degradable magnesium alloy according to claim 1, further comprising active elements, wherein the active elements include any one or a combination of two of titanium, potassium, strontium, zirconium, calcium, lithium, aluminum and manganese.

5. The fully degradable magnesium alloy according to claim 4, wherein a weight ratio of the active elements is at most 2%.

6. A method for preparing a fully degradable magnesium alloy, comprising the following steps: adding a raw material to a resistance furnace for smelting to form a smelted material under a protective gas; refining the smelted material to form a refined material; cooling after pouring the refined material to form an ingot; proceeding with solid solution treatment on the ingot prior to plastic deformation for fining alloy crystal grains; and then performing heat treatment on the alloy crystal grains to obtain a fully degradable magnesium alloy billet.

7. The method according to claim 6, wherein a smelting temperature of the raw material is 720-820° C.

8. The method according to claim 6, wherein a pouring temperature of the refined material is 700-760° C.

9. The method according to claim 6, wherein a solid solution condition of the solid solution treatment is 500-550° C. for 4-24 hours of treatment.

10. The method according to claim 6, wherein the raw material comprises magnesium, alloying elements and active elements; a weight ratio of magnesium is not less than 85%; the alloying elements comprise any one or a combination of several of gadolinium, erbium, thulium, yttrium, neodymium, holmium and zinc; the active elements comprise any one or a combination of two of titanium, potassium, strontium, zirconium, calcium, lithium, aluminum and manganese, a weight ratio of a content of the active elements is 0-2%; magnesium, aluminum and zinc are added in a form of metal, and other elements are added in a manner of an intermediate alloy.

11. The fully degradable magnesium alloy according to claims 2, further comprising active elements, wherein the active elements include any one or a combination of two of titanium, potassium, strontium, zirconium, calcium, lithium, aluminum and manganese.

12. The fully degradable magnesium alloy according to claims 3, further comprising active elements, wherein the active elements include any one or a combination of two of titanium, potassium, strontium, zirconium, calcium, lithium, aluminum and manganese.
